(19) United States
(12) Patent Application Publication
(10) Pub. No.: US 2008/0015236 A1
(43) Pub. Date: Jan. 17, 2008

(54) METHOD FOR PROMOTING MYOCARDIAL REGENERATION AND USES THEREOF

(76) Inventor: David E. Weinstein, Dobbs Ferry, NY (US)

Correspondence Address: SANDER RABIN MD JD;CONVERGENT TECHNOLOGY PATENT LAW GROUP, WHITEMAN OSTERMAN & HANNA LLP, ONE COMMERCE PLAZA, ALBANY, NY, 12260, US

(21) Appl. No.: 11/670,964

(22) Filed: Feb. 3, 2007

Related U.S. Application Data
(60) Provisional application No. 60/771,499, filed on Feb. 8, 2006.

(51) Int. Cl. A61K 31/4245

(52) U.S. Cl. 514/364

(57) ABSTRACT

The present invention provides a method for promoting myocardial regeneration in a subject. Additionally, the present invention provides a method for promoting myocardial cell and tissue regeneration in a subject. The present invention is further directed to the use of GM-284 in promoting myocardial tissue regeneration in a subject. Finally, the present invention provides a method for treating a myocardial infarction in a subject in need of such treatment.

### RELATED APPLICATION

The present invention claims priority to U.S. Provisional No. 60/771,499 filed on 8 Feb. 2006, which is incorporated herein by reference in its entirety.

### BACKGROUND OF THE INVENTION

1. Technical Field

This invention relates generally to a method for promoting the regeneration of cardiac tissue, and is particularly related to the use of the immunophilin ligand GM284 in such a method.

2. Related Art

2.10 Referenced Publications

All references cited herein, including journal articles or abstracts, published or corresponding U.S. or foreign patent applications, issued U.S. or foreign patents, or any other references, are entirely incorporated by reference herein, to disclose and describe the methods and/or materials in connection with which the publications or documents are cited, including all data, tables, figures, and text presented in the cited references. Additionally, the entire contents of the references cited within the references cited herein are also entirely incorporated by references.

Citation of any references herein is not intended as an admission that the references is pertinent prior art, or considered material to the patentability of any claim of the present application. Any statement as to content or a date of any references is based on the information available to applicant at the time of filing and does not constitute an admission as to the correctness of such a statement. The dates of publication provided may be different from the actual publication dates which may need to be independently confirmed.

Reference to known method steps, conventional methods steps, known methods or conventional methods is not in any way an admission that any aspect, description or embodiment of the present invention is disclosed, taught or suggested in the relevant art.

2.30 Tissue Injury

Injured tissues are able to heal by regeneration, by repair, or by a combination of these processes. Regeneration results in the re-establishment of the original tissue structure and function. In contrast, tissue repair results in the replacement of the original tissue with a patch of connective tissue, or scar, which is functionally inferior to the original tissue. The response of most tissues to injury falls within this spectrum.

Unlike most tissues, the heart and brain appear to differ, in that following injury, these tissues are particularly biased toward repair rather than regeneration [Sofroniew, M. V. Reactive astrocytes in neural repair and protection, Neuroscientist 11, 400-7 (2005); Anversa, P., Sussman, M. A. & Bolli, R., Molecular genetic advances in cardiovascular medicine; focus on the myocytes, Circulation 109, 2832-8 (2004)]. Evidence from a number of laboratories suggests that both the heart and the brain are capable of regeneration. However the scarring process appears to have gained a temporal advantage in these tissues following injury or disease.

The failure of the heart's myocardium to regenerate following a myocardial infarction has wide reaching consequences. It is estimated that 12 million people in the U.S. suffer from the most common form of cardiac disease, coronary heart disease (CHD), which results in approximately 1.5 million acute myocardial infarctions (MI) annually and 500,000 deaths. The combined annual cost in the U.S. for MI is in excess of $60 billion. Outside of the U.S., there are 12 million MIs per annum and about 100 million patients with CHD. The large number of affected individuals and the enormous costs associated with their care represent a tremendous unmet medical need.

2.40 Myocardial Injury

Following an MI the myocardium undergoes a stereotypical series of histopathological events that begin after about 10 to 12 minutes of myocardial anoxia. The myocardial territory served by the occluded or spastic coronary blood vessel shows a circumscribed area of ischemic necrosis, also known as coagulative necrosis. In the ensuing 12 to 48 hours, the myocardial fibers in the affected area can still be identified as such, but they lose their transversal striations and their nuclei. The interstitium is often hemorrhagic. Healing begins in earnest within 5 to 10 days. The maturing lesion is characterized by myocardial fibers with preserved contours, but their cytoplasm is intensely eosinophilic; and, both transverse striations and nuclei are completely lost the area of coagulative ischemic necrosis. The interstitium of the infarcted area is initially infiltrated, with neutrophils, which are then replaced lymphocytes and macrophages that phagocytose the myocytic debris. The necrotic area is surrounded by, and progressively invaded by, granulation tissue, which replaces the infarct with a collagenous scar. Once established, the infarct will remain in situ for the remainder of the patient's life.

There are a number of reports showing that following MI, stem cells are both mobilized and recruited into the area adjacent to the infarct [Urbanek, K. et al. Myocardial regeneration by activation of multipotent cardiac stem cells in ischemic heart failure. Proc Natl Acad Sci USA 102, 8692-7 (2005)]; or, that stem cells from a number or sources, including hematpoietic stem cells, [Glaser, R., Lu, M. M., Narula, N. & Epstein, J. A. Smooth muscle cells, but not myocytes of host origin in transplanted human hearts. Circulation 106, 17-9 (200)], mesenchymal stern cells [Fazel, S. et al. Cell transplantation preserves cardiac function after infarction by infarct stabilization: augmentation by stem cell factor. J Thorac Cardiovasc Surg 130, 1310 (2005).] or myogenic stern cells [Orlic, D. et al. Bone marrow cells regenerate infarcted myocardium. Nature 410, 701-5 (2001)] can be delivered into the infarcted site to effect partial regeneration of the heart. Taken together, these observations suggest that there are stem cell pools that can potentially be activated to regenerate cardiac tissue following an MI.

The inventor herein demonstrates that daily treatment with GM284, a non immunosuppressive immunophilin ligand, beginning after an MI has occurred, results in the revascularization of the infracted territory and the regeneration of cardiac myocytes. This is the first disclosure of a pharmacological intervention resulting in the regeneration of mammalian cardiac tissue following MI.

The inventor has previously shown that GM284 promotes rapid and extensive regeneration of a number of organ systems following injury, including the peripheral nervous system, the dermis and epidermis; and, more recently, composite tissues such as those present in the ear. In all of these tissues, GM284 accelerates naturally occurring regenerating systems through a series of molecular and cellular interactions, including the up-regulation of transcription factors known to be critical in mediating regeneration [Gondré, M., Burrola, P. & Weinstein, D. E. Accelerated nerve regeneration mediated by Schwann cells expressing a mutant form of the POU protein SCIP. J Cell Biol 141, 493-501 (1998); Weinstein, D. E. The Role of Schwann cells in Neural Regeneration. The Neuroscientist 5, 208-216 (1999)]. Moreover, in these systems, GM284 accelerates cell-cell interactions that mediate regeneration. Histological analysis suggests that GM284 also accelerates cell-cell interactions that mediate regeneration following an MI.

The regeneration of complex tissues and entire body parts are phenomena that are shared by many lower species. For instance, starfish regenerate entire limbs after amputations and the snail can regenerate its head. In contrast, most vertebrates have lost the ability to regenerate entire body parts. It is not clear whether the evolutionary “loss” of complex tissue regeneration in vertebrates represents a true inability to re-form these tissues following injury or whether the molecular and cellular mechanisms that control the processes have been re-directed or used in other organic biologies. The data presented herein argue for the latter. The inventor believes that GM284 activates a cascade of interactions that lock tissues in a state of regeneration. Moreover, the inventor's data suggest that these events are conserved across evolutionary lines, and GM284 takes advantage of their existence in mammals.

### 3.0 SUMMARY OF THE INVENTION

The present invention is based on the inventor's discovery that GM284, beginning in the hours following acute ischemic myocardial infarction in the rat, and given daily for two weeks, results in the initiation of robust cardiac regeneration, as evidenced by neovascularization of the infarcted area, and the identification of regenerating myocardial cells within the region of myocardium that includes a myocardial infarction. This novel utility of GM284 is substantial, specific, credible and unexpected.

Consequently, the preferred embodiments of present invention provide;
1) a method for promoting myocardial regeneration in a subject, by administering to the subject an amount of GM284 effective to promote myocardial regeneration of in the subject;
2) a use of GM284 to promote myocardial regeneration in a subject, wherein the GM284 is administered to the subject in an amount effective to promote regeneration of myocardium in the subject;
3) a method for promoting myocardial tissue regeneration in a subject, by administering to the subject an amount of GM284 effective to promote regeneration of myocardial, tissue in the subject;
4) a use of GM284 to promote myocardial tissue regeneration in a subject, the GM284 is administered to the subject in an amount effective to promote regeneration of myocardial tissue in the subject;
5) a method for promoting myocardial cell regeneration, by contacting myocardial tissue with an amount of GM284 effective to promote myocardial cell regeneration;
6) a use of GM284 to promote myocardial cell regeneration, wherein myocardial tissue is contacted with an amount of GM284 effective to promote myocardial cell regeneration;
7) a method for treating a myocardial infarction in a subject in need of treatment therefore, by administering by administering to the subject an amount of GM284 effective to treat the myocardial infarction in the subject;
8) a use of GM284 to treat a myocardial infarction in a subject in need of treatment therefore, by administering by administering to the subject an amount of GM284 effective to treat the myocardial infarction in the subject.

Additional aspects of the present invention will be apparent in view of the description that follows.

### 4.0 BRIEF DESCRIPTION OF THE FIGURES

FIG. 1 is an illustration of GM284.

FIG. 2 shows two low-power (FIG. 2a and FIG. 2b), and one high-power (FIG. 2c) fixed, paraffin-embedded micrographs of rat cardiac tissue harvested two weeks after occlusion of the left anterior descending (LAD) coronary artery with and without daily treatment with GM284, FIG. 2a shows harvested cardiac tissue treated with a vehicle. FIG. 2b shows harvested cardiac tissue treated with GM284. FIG. 2b demonstrates that treatment with GM284 results in an increase in viable myocardial cells and an increase in the intramural vascularity of the harvested cardiac tissue. FIG. 2c shows the myocardium of a rat 2 weeks after occlusion of the LAD and treatment with GM284, with newly formed cardiomyocytes, extravasating cells, and a large central intramural blood vessel.

FIG. 3 shows two low-power (FIG. 3a and FIG. 3b), and three higher-power (FIGS. 3c-3e) fixed, paraffin-embedded micrographs of rat cardiac tissue harvested four weeks after occlusion of the left anterior descending coronary artery (LAD) and daily treatment with or without GM284. FIG. 3a (trichrome stain) shows that there is virtually no scarring in GM284-treated cardiac tissue. FIG. 3b (trichrome stain) shows an extensive area of infarction in the vehicle-treated cardiac tissue. FIG. 3c (H&E stain) shows normal cardiac histology in GM284-treated cardiac tissue. FIG. 3d (H&E stain) shows extensive fibrosis in the vehicle-treated cardiac tissue. FIG. 3e (H&E stain) shows human cardiac tissue one month following a myocardial infarction, which is similar to FIG. 3d.

### 5.0 DETAILED DESCRIPTION OF THE INVENTION

The following detailed description illustrates the invention by way of example, not by way of limitation of the principles of the invention. This description will clearly enable one skilled in the art to make and use the invention, and describes several embodiments, adaptations, variations, alternatives and uses of the invention, including what we presently believe is the best mode of carrying out the invention. It is to be understood that this invention is not limited to the particular embodiments described, as such may, of course, vary.

5.10 Lexicon

It is to be understood that the terminology used herein is for the purpose of describing particular embodiments only, and is not intended to be limiting, since the scope of the present invention will be limited only by the appended claims.

As used herein and in the appended claims, the singular indefinite forms “a”, “an”, and the singular definite form “the” include plural referents unless the context clearly dictates otherwise. Thus, for example, reference to “a domain” includes a plurality of such domains and reference to “an energy state” includes reference to one or more energy states and equivalents thereof known to those skilled in the art, and so forth.

As used herein, the term “promoting regeneration of myocardial tissue” means augmenting, improving, increasing, or inducing partial or full growth or regrowth of myocardial tissue in a region of myocardium that includes a myocardial infarction.

As used herein, the term “growth” or “regrowth” refers to an increase in mass, volume, and/or thickness of myocardial tissue, and includes an increase in myocardial cell proliferation.

As used herein, the phrase “promote regeneration of infarcted myocardium” means effective to ameliorate or moderate the clinical impairment or clinical symptoms associated with a myocardial infarction. For example, in a myocardial infarction, the associated clinical impairment or symptoms may be ameliorated or moderated by:
1) inducing or accelerating the regeneration of myocardial cells in a region of myocardium that includes a myocardial infarction;
2) inducing or accelerating the regeneration of infarcted myocardial tissue;
3) inducing or accelerating proliferation of epicardial tissue over the myocardial infarction;
4) inducing or accelerating proliferation of endocardial tissue over the myocardial infarction.

The inventor has discovered that a nonimmunosuppressive immunophilin ligand, GM284, that is known to enhance axonal regeneration and induce hypermyelination following mechanical transection of peripheral nerves, promotes the regeneration of infarcted myocardial tissues.

5.20 GM284

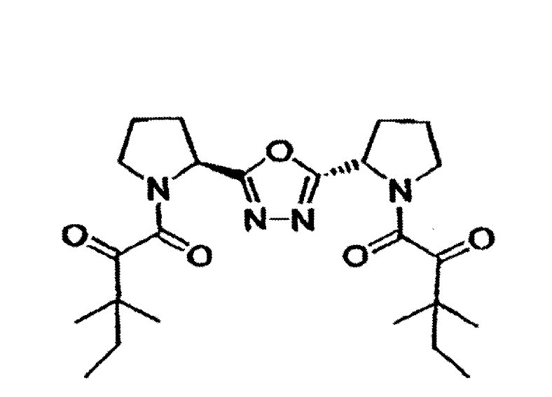

The structure of GM284 is depicted m FIG. 1. U.S. Pat. No. 6,809,107, entitled, “Neurotrophic pyrrolidines and piperidines, and related compositions and methods,” issued to Kanojia. et al, on Oct. 26, 2004, and assigned to Ortho-McNeil Pharmaceutical, Inc. (“Ortho Patent '107”), which is herein incorporated by reference, discloses the composition and structure of GM284, GM284 may be prepared in accordance with method described in Ortho Patent '107.

It is anticipated that GM284 will be effective as a drug to treat myocardial infarction, as well as many types of disorders associated with myocardial tissue degeneration. U.S. patent application Ser. No. 10/290,657, entitled, “Methods for promoting wound healing and uses thereof” filed by the present inventor on Nov. 8, 2002, which is herein incorporated by reference, discloses the use of GM284 for regenerating cardiac tissues such as endocardial and epicardial tissues.

5.30 GM284 and Myocardial Regeneration

In the course of his experiments, the inventor discovered that the infarcted myocardium of animals treated systemically with GM284 appeared dramatically different from those of vehicle-treated animals. The inventor's analysis of his experiments demonstrates that the infarcted myocardium of rats treated with GM284 regenerated, as opposed to undergoing scar formation, compared with controls.

Accordingly, the present invention provides a method for promoting myocardial tissue regeneration in a subject in need of such regeneration. As demonstrated herein, the immunophilin ligand, GM284, has the ability to promote healing of a myocardial infarction by promoting myocardial tissue regeneration in the region of myocardium that includes a myocardial infarction; and/or by enhancing proliferation of endocardial and epicardial tissues in the in the region of myocardium that includes a myocardial infarction.

The amount of GM284 effective to promote healing of a myocardial infarction in a subject in need thereof will vary depending upon the particular factors of each case, including the location and size of the myocardial infarction, the severity of the myocardial infarction, the length of time to treatment, and the method of administration. This amount may be readily determined by the skilled artisan, based upon known procedures, including clinical trials, and methods disclosed herein.

Regeneration or enhanced regeneration of myocardial tissue in a region of the myocardium that includes a myocardial infarction may be promoted, for example, by enhancing regeneration of myocardial cells in the region of the myocardium that includes the myocardial infarction. In a subject, the regeneration of myocardial tissue is promoted in the region of myocardium that includes a myocardial infarction in the subject; and, thus, contributes to the promotion of healing of the myocardial infarction in the subject.

The myocardial infarction may be the result of myocardial ischemia or any affliction (e.g., disease, injury, surgery) that eventuates in a myocardial infarction, such as, for example, and without limitation, coronary artery occlusion, coronary artery vasospasm, blunt trauma to the chest, hemopericardium, pericardial effusion, pericarditis, endocarditis, myocarditis, epicarditis, cardiac valvular disease, hypotension, hemorrhage, or a blood dyserasia. The subject may be any animal, but is preferably a mammal (e.g., humans, domestic animals and commercial animals). More preferably, the subject is a human.

In the method of the present invention, GM284 is administered to a subject in an amount effective to promote regeneration of infarcted myocardium in the subject.

In the present invention, the effective amount of GM284 is between about 1 mg/kg and about 10 mg kg or between about 0.1 pM and about 5 mM.

The method of the present invention may be used, to promote myocardial regeneration in a subject includes the step of administering GM284 to the subject. The GM284 is administered to the subject in an amount effective to promote myocardial tissue regeneration in the subject, as defined above. In the present invention, the effective amount of GM284 is between about 1 mg/kg and about 10 mg/kg or between about 0.1 pM and about 5 mM.

The method of the present invention comprises contacting myocardial tissue with GM284. The myocardial tissue may be damaged or healthy/undamaged. The myocardial tissue may comprise a region of myocardium that includes a myocardial infarction. The GM284 is contacted with myocardial tissue in an amount effective to promote regeneration of at least one myocardial cell. This amount may be determined by the skilled artisan using known procedures (e.g., concentration curves, ELISA, protein-concentration determination, radioimmunoassay, titration curves, and methods disclosed herein.)

The method of the present invention, may be used to promote regeneration of at least one myocardial cell in vitro, or in vivo in a subject. For example, GM284 may be contacted in vitro with myocardial tissue (e.g., a biopsy or plug of myocardial tissue removed from a subject) by introducing GM284 to the tissue using conventional procedures. Alternatively, GM284 may be contacted in vivo with myocardial tissue in a subject by administering GM284 to the subject.

It is also within the scope of the present invention that GM284 may be introduced to myocardial tissue in vitro, using conventional procedures, to promote regeneration of myocardial cells in vitro. Thereafter, myocardial tissue containing myocardial cells may be introduced into a subject to provide myocardial cells in vivo. In such an ex vivo approach, the myocardial tissue is preferably removed from the subject, subjected to introduction of GM284, and then reintroduced into the subject. The myocardial cell regeneration promotes healing of a myocardial infarction in the subject.

Accordingly, the present invention provides a method for treating a myocardial infarction in a subject in need of treatment, comprising contacting myocardial tissue in the subject with GM284 (e.g., by administering GM284 to the subject), thereby treating the myocardial infarction. Myocardial infarctions that may be treated by methods disclosed herein include disorders characterized by infarction of myocardial cells. The GM284 is contacted with myocardial tissue in a subject (e.g., administered to a subject), for the purpose of treating a myocardial infarction, in an amount effective to promote regeneration of at least one myocardial cell.

According to the method of the present invention, GM284 may be administered to a human or animal subject by known procedures, including, without limitation, oral administration, parenteral administration (e.g., epifascial, intracapsular, intracutaneous, intradermal, intramuscular, intraorbital, intraperitoneal, intraspinal, intrasternal, intrathecal, intravascular, intravenous, parenchymatous, or subcutaneous administration), sublingual administration, topical administration, transdermal administration, and administration through an osmotic mini-pump. Preferably, the immunophilin ligand is administered topically.

For oral administration, the formulation of the immunophilin ligand may be presented as capsules, tablets, powders, granules, or as a suspension. The formulation may have conventional additives, such as lactose, mannitol, cornstarch, or potato starch. The formulation also may be presented with binders, such as crystalline cellulose, cellulose derivatives, acacia, cornstarch, or gelatins. Additionally, the formulation may be presented with disintegrators, such as cornstarch, potato starch, or sodium carboxymethylcellulose. The formulation also may be presented with dibasic calcium phosphate anhydrous or sodium starch glycolate. Finally, the formulation may be presented with lubricants, such as talc or magnesium stearate.

For parenteral administration (i.e., administration by injection through a route other than the alimentary canal), the immunophilin ligand may be combined with a sterile aqueous solution that is preferably isotonic with the blood of the subject. Such a formulation may be prepared by dissolving a solid active ingredient in water containing physiologically-compatible substances, such as sodium chloride, glycine, and the like, and having a buffered pH compatible with physiological conditions, so as to produce an aqueous solution, then rendering said solution sterile. The formulations may be presented in unit or multi-dose containers, such as sealed ampoules or vials. The formulation may be delivered by any mode of injection, including, without limitation, epifascial, intracapsular, intracranial, intracutaneous, intramuscular, intraorbital, intraperitoneal, intraspinal, intrasternal, intrathecal, intravascular, intravenous, parenchymatous, or subcutaneous.

For transdermal administration, the immunophilin ligand may be combined with skin penetration enhancers, such as propylene glycol, polyethylene glycol, isopropanol, ethanol, oleic acid, N-methylpyrrolidone, dimethyl sulfoxide, and the like, which increase the permeability of the skin to the immunophilin ligand, and permit the immunophilin ligand to penetrate through the skin and into the bloodstream. The ligand/enhancer compositions also may be further combined with a polymeric substance, such as ethylcellulose, hydroxy-propyl cellulose, ethylene/vinylacetate, polyvinyl pyrrolidone, and the like, to provide the composition in gel form, which may be dissolved in solvent, such as methylene chloride, evaporated to the desired viscosity, and then applied to backing material to provide a patch. The immunophilin ligand may be administered transdermally at the site of the wound in the subject where neural trauma has occurred, or where the wound is localized. Alternatively, the immunophilin ligand may be administered transdermally at a site other than the affected area, in order to achieve systemic administration.

For topical administration, the immunophilin ligand may be combined with additional materials that are known for use in skin-care products, or which are otherwise suitable for topical application. Such optional materials include, but are not limited to, disbursing agents, masking agents, preservatives, processing agents, and additives having specific physicochemical properties, such, as polymeric film formers and the like.

GM284 may also be released or delivered from an osmotic mini-pump or other time-release device. The release rate from an elementary osmotic mini-pump may be modulated with a microporous, fast-response gel disposed in the release orifice. An osmotic mini-pump would be useful for controlling release, or targeting delivery, of the immunophilin ligand.

It is within the scope of the present invention that a formulation containing GM284 may be farmer associated with a pharmaceutically acceptable carrier, thereby comprising a pharmaceutical composition. Accordingly, the present invention further provides a pharmaceutical composition, comprising GM284 and a pharmaceutically acceptable carrier. The pharmaceutically acceptable carrier must be “acceptable” in the sense of being compatible with the other ingredients of the composition, and not deleterious to the recipient thereof. Examples of acceptable pharmaceutical carriers include carboxymethylcellulose, crystalline cellulose, glycerin, gum arable, lactose, magnesium stearate, methyl cellulose, powders, saline, sodium alginate, sucrose, starch, talc, and water, among others. Formulations of the pharmaceutical composition may be conveniently presented in unit dosage.

The formulations of the present invention may be prepared by methods well known in the pharmaceutical arts. For example, GM284 may be brought into association with a carrier or diluent, as a suspension or solution. Optionally, one or more accessory ingredients (e.g., buffers, flavoring agents, surface active agents, and the like) also may be added. The choice of carrier will depend upon the route of administration. The pharmaceutical composition would be useful for administering the GM284 of the present invention to a subject to promote healing of a wound. The GM284 is provided in an amount that is effective to promote wound healing in a subject to whom the pharmaceutical composition is administered. That amount may be readily determined by the skilled artisan, as described above.

The present invention also provides a method for promoting regeneration of myocardial tissue in a subject. As described above, regeneration of myocardial tissue in a subject may be promoted by enhancing proliferation of myocardial cells in the subject. Accordingly, in one embodiment of the invention, the regeneration of myocardial tissue is promoted at the site of a myocardial infarction in the subject, and, thus, contributes to the promotion of healing of the myocardial infarction in the subject.

The present invention is described the following example, which is set forth to aid in the understanding of the invention, and should not be construed to limit in any way the scope of the invention as defined in the claims which follow thereafter.

### EXAMPLE

### GM284 Promotes Myocardial Cell Regeneration

The left anterior descending artery (“LAD”) of 36 male adult Sprague Dawley rats weighing between 250 and 270 grams was occluded by placement of a ligature around the LAD and pulling it taught. The ligature was left in place to create a chronic ischemic area (myocardial infarction) distal to the occlusion. Following the closure of the chest and evacuation of the extra-pulmonary air to reverse the surgery-induced pneumothorax, the rats were returned to their cages for recovery, where they had ad libitum access to water and food for the duration of the experiment. Immediately following surgery the rats received the analgesic Buprenex at a dose of 1.5 mg/kg subcutaneously every 12 hours for the first 72 hours, and then on an as-needed basis.

The GM284 was dissolved to a final concentration of 5 mg/kg in phosphate buffered saline (“PBS”); 10 μM stock solution of GM284 in dimethyl sulfoxide (“DMSO”) was diluted in PBS. The vehicle was 250 μl of DMSO into 10 ml PBS.

Postsurgically, the rats were randomly assigned into six (6) experimental groups (A through F) with 6 animals in each group (n=36). “Vehicle” groups A, C and E received daily intraperitoneal injections of vehicle; and, “treatment” groups B, D, and F were treated with intraperitoneal injections of GM284.

Animals in treatment groups B, D, and F received daily intraperitoneal injections of GM284 at 5 mg/kg; and, animals in vehicle groups A, C and E received daily intraperitoneal injections of only the vehicle in which the GM284 was dissolved. The treatment group animals had a 100% survival over the length of the study, in contrast, there was a mortality of 25% over the course of the study in the vehicle treated group.

Animals in vehicle group A and treatment group B were treated for 2 weeks after LAD ligation and sacrificed.

Animals in vehicle group C and treatment group D were treated for 2 weeks following LAD ligation. Thereafter, the vehicle or GM284 injections were withdrawn and the animals were allowed to survive for an additional 2 weeks prior to sacrifice.

Animals in vehicle group E treatment group F were treated daily for 30 days following LAD ligation and then sacrificed.

Prior to sacrifice, the animals were anesthetized the hearts were harvested and processed for histological analysis by submersion-fixing in 4% paraformaldehyde at 4° C. overnight, followed by dehydration and embedding in paraffin. Eight-micra tissue sections were cut through the infarcted area and stained with H&E and trichrome. The histological sections were viewed by light microscopy, by a reviewer blind to the treatment groups, and evaluated for:
1) the size of the infarct;
2) the presence of viable cardiac myocytes; and,
3) the extent of collateral vascularity.

Histological analysis of the hearts two weeks following induction of myocardial ischemia revealed clear differences in the affected regions of animals that received GM284 in contrast to analogous regions of the vehicle-treated rat hearts. The myocardia of the vehicle-treated animals underwent the middle to late stages of scarification, demonstrating leukocyte infiltration, atrophied cardiac myocytes and a few isolated intact myocytes.

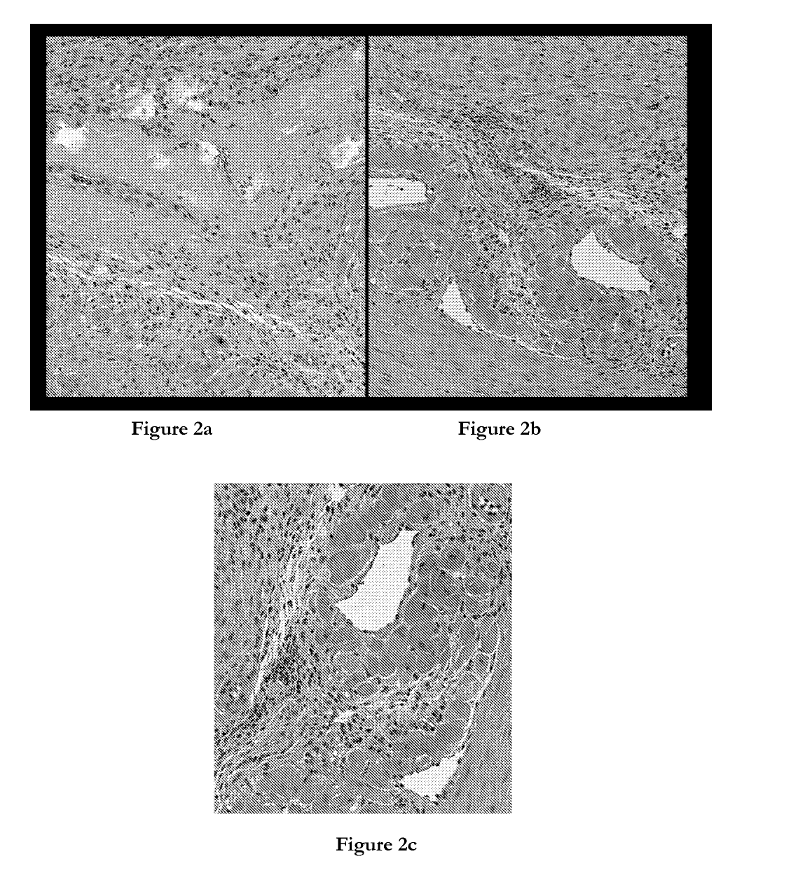

FIG. 2 shows two low-power (FIG. 2a and FIG. 2b), and one high-power (FIG. 2c) fixed, paraffin-embedded micrographs of rat cardiac tissue harvested two weeks after occlusion of the left anterior descending (LAD) coronary artery with and without daily treatment with GM284, FIG. 2a shows harvested cardiac tissue treated with a vehicle. FIG. 2b shows harvested cardiac tissue treated with GM284. FIG. 2b demonstrates that treatment with GM284 results in an increase in viable myocardial cells and an increase in the intramural vascularity of the harvested cardiac tissue.

In FIG. 2a, the region of myocardium that includes a myocardial infarction in vehicle-treated animals shows a resolving infarct with extensive fibrosis and a large leukocytic infiltrate. The few surviving cardiac myocytes within the affected area appear atrophic.

In contrast to FIG. 2a, the myocardium in the affected areas of the GM284-treated rats shown in FIG. 2b, appears to either have been spared or to have undergone active regeneration. Unlike the sparse number of surviving myocytes in the vehicle-treated rats (FIG. 2a) there are a numerous intact myocytes in FIG. 2b. FIG. 2b shows the presence of large intramural vessels and smaller arterioles that appear” to be part of a neovascularization process. In addition, in FIG. 2b, there are numerous collateral vessels in the GM284-treated cardiac tissues that are rare in the corresponding vehicle treated tissue of FIG. 2a. The presence of the large number of collateral vessels in the GM284-treated tissue suggests that GM284 either promotes vasculogenesis or promotes the opening of quiescent vessels that are resident but non-patent in the homeostatic heart.

FIG. 2c shows the myocardium of a rat 2 weeks after occlusion of the LAD and treatment with GM284, with newly formed cardiomyocytes, extravasating cells, and a large central intramural blood vessel. Increased magnification of the GM284-treated heart in FIG. 2c demonstrates streams of cells cascading through the infracted area that, when viewed at higher power can be seen to articulate with the apical aspect of the vessel. The apposition of the stream of cells to the vessel suggests that they are blood-borne cells extravasating and possibly contributing to cardiac regeneration. In FIG. 2b and FIG. 2c, the myocardial cells are seen to be plump with prominent nuclei. The myocardial cells are arrayed in annuli around the vessels.

In an effort to determine the extent of the pro-regenerative effects of GM284 following cardiac ischemia, animals in vehicle group E and treatment group F were followed for 30 days. Over the month following the induction of ischemia, the fibrotic region of the vehicle-treated animals in group E matured fully.

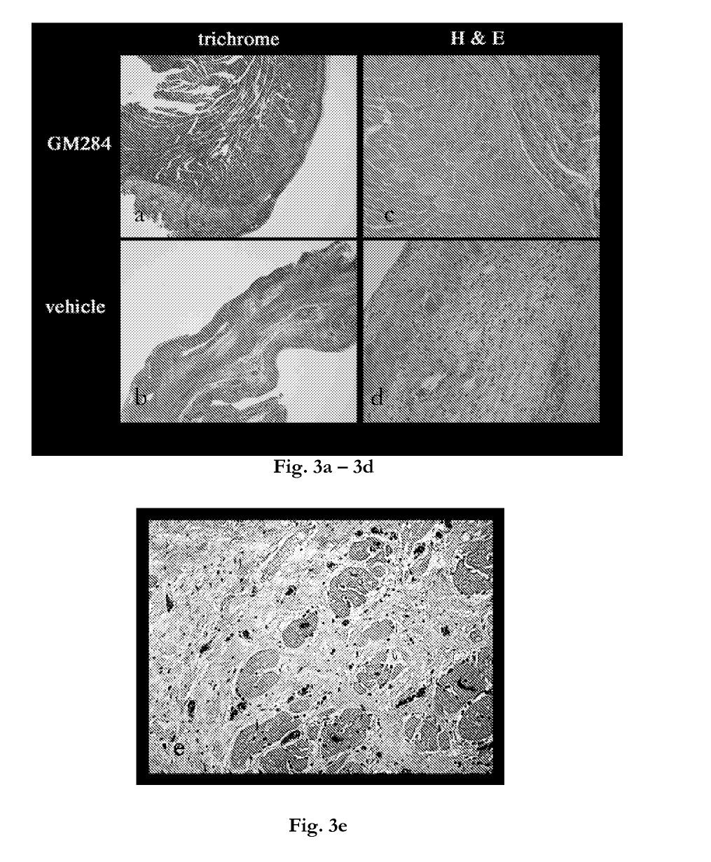

FIG. 3 shows two low-power (FIG. 3a and FIG. 3b), and three higher-power (FIGS. 3c-3e) fixed, paraffin-embedded micrographs of rat cardiac tissue harvested four weeks after occlusion of the left, anterior descending coronary artery (LAD) and daily treatment with or without GM284. FIG. 3a (trichrome stain) shows that there is virtually no scarring in GM284-treated cardiac tissue. FIG. 3b (trichrome stain) shows an extensive area of infarction in the vehicle-treated cardiac tissue. FIG. 3c (H&E stain) shows normal cardiac histology in GM284-treated cardiac tissue, FIG. 3d (H&E stain) shows extensive fibrosis in the vehicle-treated cardiac tissue. FIG. 3e (H&E stain) shows human cardiac tissue one month following a myocardial infarction, which is similar to FIG. 3d.

The group E vehicle-treated tissue shown in FIG. 3b has extensive scarring with small regions of cardiac myocytes scattered in small islands throughout the infarct. In contrast, the group F GM284-treated tissue shown in FIG. 3a reveals minimal fibrosis scattered among otherwise intact, apparently healthy muscle. The ischemic region of the vehicle-treated tissue of FIG. 3d shows extensive loss of cardiac muscle, similar to human heart one month following an extensive MI, as shown in FIG. 3e; while, the GM284-treated tissue has healthy, intact cardiac myocytes as shown in FIG. 3c.

Accordingly, the treatment of animals with GM284 for two weeks and allowing them to survive for an additional two weeks prior to sacrifice did not significantly alter the histological appearance of the cardiac tissue, in comparison to the animals that were analyzed immediately at the cessation of treatment in contrast the cardiac infarcts in the vehicle-treated rats continued to mature and fibrose into a stable, rigid structure.

This histological analysis raises the likelihood that GM284 acts to accelerate cell-cell interactions that mediate regeneration.

While the foregoing invention has been described in some detail for purposes of clarity and understanding, it will be appreciated by one skilled in die art, from a reading of the disclosure that various changes in form and detail can be made without departing from the true scope of the invention in the appended claims.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention.

While this invention has been described, in connection with specific embodiments thereof, it will be understood that it is capable of further uses, variations modifications or adaptations. Such uses, variations, modifications and adaptations are intended to be within the meaning and range of equivalents of the disclosed embodiments, based on the teaching and guidance presented herein.

Having fully described this invention, it will also be appreciated by those skilled in the art that the same can be performed within a wide range of equivalent parameters, concentrations, and conditions without departing from the spirit and scope of the invention and without undue experimentation.

It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

It is believed that the disclosure set forth above encompasses multiple distinct inventions with independent utility. While each of these inventions has been disclosed in its preferred form, the specific embodiments thereof as disclosed and illustrated herein are not to be considered in a limiting sense as numerous variations are possible.

The subject matter of the inventions includes all novel and non-obvious combinations and subcombinations of the various elements, features, functions and/or properties disclosed herein.

No single feature, function, element or property of the disclosed embodiments is essential to all of the disclosed inventions. Similarly, where the claims recite “a” or “a first” element or the equivalent thereof, such claims should be understood to include incorporation of one or more such elements, neither requiring nor excluding two or more such elements.

It is believed that the following claims particularly point out certain combinations and subcombinations that are directed to one of the disclosed inventions and are novel and non-obvious. Inventions embodied in other combinations and subcombinations of features, functions, elements and/or properties may be claimed through amendment of the present claims or presentation of new claims in this or a related application.

Such amended or new claims, whether they are directed to a different invention or directed to the same invention, whether different, broader, narrower or equal in scope to the original claims, are also regarded as included within the subject matter of the inventions of the present disclosure.

What is claimed is:

1. A method for promoting myocardial regeneration in a subject, comprising the step of administering to the subject an amount of GM284 effective to promote myocardial regeneration in the subject.

2. The method of claim 1, wherein the myocardial regeneration is promoted in the subject by promoting myocardial tissue regeneration.

3. The method of claim 2, wherein the myocardial tissue regeneration in the subject is promoted by regenerating myocardial cells.

4. The method of claim 1, wherein the subject is a mammal.

5. The method of claim 4, wherein the mammal is a human.

6. The method of claim 1, wherein the amount of GM-284 is between about 1 mg/kg and about 10 mg/kg.

7. The method of claim 6, wherein the amount of GM-284 is about 5 mg/kg.

8. The method of claim 1, wherein the amount of GM-284 is between about 0.1 pM and about 5 mM.

9. The method of claim 8, wherein the amount of GM-284 is between about 5 pM and about 1.5 mM.

10. The method of claim 1, wherein the GM-284 is administered to the subject by oral administration, parenteral administration, sublingual administration, topical administration, transdermal administration, or osmotic pump.

11. A method for promoting myocardial tissue regeneration in a subject, comprising the step of administering to the subject an amount of GM-284 effective to promote myocardial tissue regeneration in the subject.

12. The method of claim 11, wherein the myocardial tissue regeneration in the subject is promoted by promoting myocardial cell regeneration in the subject.

13. The method of claim 12, wherein the myocardial cell regeneration is promoted in the region of a myocardial infarction in the subject.

14. The method of claim 13, wherein the subject is a mammal.

15. The method of claim 13, wherein the mammal is a human.

16. The method of claim 13, wherein the amount of GM-284 is between about 1 mg/kg and about 10 mg/kg.

17. The method of claim 16, wherein the amount of GM-284 is about 5 mg/kg.

18. The method of claim 13, wherein the amount of GM-284 is between about 0.1 pM and about 5 mM.

19. The method of claim 18, wherein the amount of GM-284 is between about 5 pM and about 1.5 mM.

20. The method of claim 13, wherein the GM-284 is administered to the subject by oral administration, parenteral administration, sublingual administration, topical administration, transdermal administration, or osmotic pump.

21. A method for promoting myocardial cell regeneration, comprising contacting myocardial tissue with an amount of GM-284 effective to promote myocardial cell regeneration.

22. The method of claim 21, wherein the contacting is effected in vitro.

23. The method of claim 21, wherein the contacting is effected in vivo in a subject.

24. The method of claim 23, wherein the myocardial cell regeneration promotes healing of a myocardial infarction in the subject.

25. The method of claim 23, wherein the contacting is effected in vivo in a subject by administering GM-284 to the subject.

26. The method of claim 25, wherein the GM-284 is administered to the subject by oral administration, parenteral administration, sublingual administration, topical administration, transdermal administration, or osmotic pump.

27. The method of claim 25, wherein the subject is a human.

28. The method of claim 27, wherein the human has a myocardial infarction.

29. The method of claim 26, wherein the amount of the immunophilin ligand is between about 0.1 pM and about 5 mM.

30. The method of claim 29, wherein the amount, of die immunophilin ligand is between about 5 pM and about 1.5 mM.

31. A method for treating a myocardial infarction in a subject in need of treatment, comprising administering to the subject an amount, of GM-284 effective to treat the myocardial infarction in the subject.
